(19) United States
(12) Patent Application Publication
(10) Pub. No.: US 2014/0041672 A1
(43) Pub. Date: Feb. 13, 2014

(54) FIXER OCCLUDER PENILE DEVICE FOR URINARY INCONTINENCE

(71) Applicant: PEDRO GARCÍA BERRUEZO, TORRE DEL MAR (MALAGA) (ES)

(72) Inventor: PEDRO GARCÍA BERRUEZO, TORRE DEL MAR (MALAGA) (ES)

(21) Appl. No.: 13/949,532

(22) Filed: Jul. 24, 2013

(30) Foreign Application Priority Data
Aug. 10, 2012 (ES) ES201200849

(51) Int. Cl. A61F 2/00

(52) U.S. Cl. CPC A61F 2/0054
USPC 128/885

(57) ABSTRACT

The fixer occluder penile device for urinary incontinence is a flattened ring of soft material that covers an internal metal structure. It is deformable, adjustable and has two semicircular bulges in the top inside and a bulge elongate on the bottom.

### 1. TECHNICAL SECTOR

It is a medical device that fits in the orthopedic industry, specifically belongs to the branch of urology.

### 2. BACKGROUND OF THE INVENTION (PREVIOUS TECHNOLOGY)

The fixer occluder penile device for urinary incontinence for which this patent is requested, emerges to solve the problem of existing devices:

1) Devices which are closed with a Velcro, which have to be dismantled whenever the patient is going to urinate, can rotate around the penis and consequently can lose their pressure on the urethra.

2) Devices which are the type of clamp, in which the penis is displaced laterally and also, involuntary urine output occurs.

These are the above patents which are related to this device:
ES P200802479 (PEDRO GARCIA BERRUEZO) Feb. 28, 2012.
WO 2007130977 A1 (GT UROLOGICAL LLC et al.) Of Nov. 15, 2007, page 4, lines 14-18, page 8, lines 15-22, page 22, line 26, page 23, line 3, page 25, line 1-13; 9A-11B.
WO 2005110279 A2 (NOVATEK MEDICAL LLC; TIMM et al.) Of Nov. 24, 2005, page 3, lines 14-30, page 4, lines 6-14, page 5, lines 15-17, page 9, lines 12 - 21, page 14, line 7, page 16, line 16, FIGS. 8A-9.
DE 10213452 C1 (YONYE ADAM) Jul. 17, 2003, abstract; figures.
U.S. Pat. No. 6,349,727 B1 (POS-T-VAC INC.). Feb. 26, 2002, column 1, line 43, column 2, line 16, column 4, lines 34-59, Figs.
DE 20112864 U1 (UROVISION GMBH) Nov. 22, 2001, abstract; figures.
U.S. Pat. No. 3,147,754 A (KOESSLER) Aug. 9, 1964, column 3, lines 30-50, Figs.
U.S. Pat. No. 714,850 A (ZIMMERMANN) Dec. 2, 1902, lines 10-48, Figs.

### 3. DESCRIPTION OF THE INVENTION

### A) Brief Summary of the Invention

Consists of a flexible metal structure coated by injection of thermoplastic or silicone rubber, with two semicircular bulges on the top inside of device. These upper bulges do that the lower bulge can exert most pressure on the urethra for controlling urinary incontinence.

### B) Detailed Description of the Invention

The fixer occluder penile device for urinary incontinence is a ring of soft material which, when it is pressed laterally, becomes oval.

Their internal structure is formed by two curved strips of mild steel and articulated at the ends by two hinges with small pins which allow the change in flattened shape to oval by simple lateral pressure with two fingers.

The metal structure has an electrolytic bath of stainless.

Also the device has two spaces free in the sides, which allow passage of blood flow.

For pressure regulation required by each patient, has two holes in which are inserted small cylinders of different diameters.

### 4. DESCRIPTION OF THE SEVERAL VIEWS OF THE DRAWING

FIG. 1 is the isometric from the lower perspective in which the number 1 is the total length of 91 mm. The number 2 is the width of 20 mm and the number 3 is the height of 20 mm.

FIG. 2 is the front view of the device in which the metal structure is represented internally. No. 4 is the thickness of the over-injected soft materials and which is of 5 mm, with all smoothed edges, as indicated by No. 8. No. 5 are the two upper semicircles for centering and fixing the penis. These semicircles have 4 mm diameter, 20 mm long and the distance between their centers is 46 mm. No. 6 is the bottom semicircular bulge of length equal to the upper space to stay centered and make optimum pressure on the urethra, has a length of 46 mm and a height of 5.5 mm, leaving a free space of 4.5 mm up to the top part. No. 7 corresponds to the two free spaces that facilitate blood circulation and penis skin gets in them. No. 9 is the metal internal structure profile. No. 10 is joints of hinges of the metal structure for rotate very well and the device can be transformed into oval. No. 11 are holes of 2.5 mm in diameter in which each patient inserted pairs of tubes of 4, 5 or 6 mm in diameter and 15 mm long to give a greater or lesser pressure on the urethra.

FIG. 3 is another isometric view from the top which shows internal metal structure and the soft coating with smoothed edges. No. 12 is the inner metal structure itself which consisting of two curved strips of mild steel of 15 mm wide and 0.25 mm thick, which is articulated at the ends by two pin hinges. The longitudinal distance between hinge is 80 mm, the central height 15, 5 mm. The distance between the two holes for the regulators is 77.5 mm. The numbers 13 and 14 indicate the place where the patient places two fingers to press and the device turns into an oval shape.

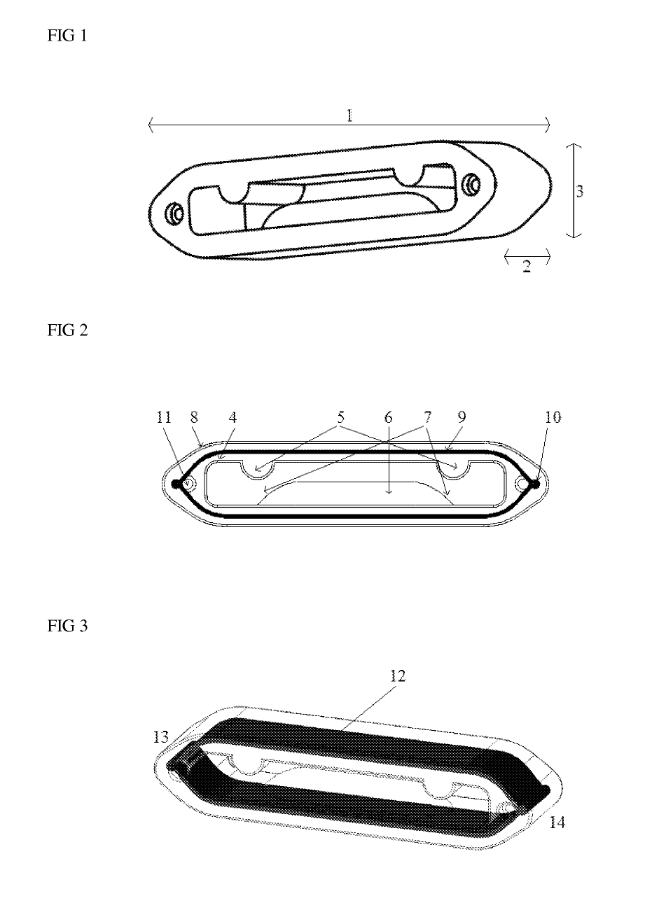

### 5. EXAMPLE OF MANUFACTURE OF THE INVENTION

The occluder fixer penile device for urinary incontinence comprises an inner metal structure consisting of two tempered steel strips which are hinged at the ends by two hinges with pins. These strips mentioned above in describing the drawings have 15 mm wide by 0.25 thick and are hinged together at a distance of 80 mm. This metallic structure is coated with electrolytic matt nickel of 10 microns thick than makes steel totally stainless.

This structure bears a coating of thermoplastic over injected or silicone rubber medical, with certification USP Class VI (because it is in contact with the skin) with a thickness of 5 mm for all the thinner parts, giving an absolute safety since this thick avoids, in a hypothetical breakage of the inner metal structure, the output of broken metal structure.

In the explanation of the drawings are absolutely all measures of the device.

Note that all edges are rounded to avoid discomfort, which does not happen with the existing ones.

A manufacturing process should incorporate the following:
1. 3D technology of graphical design.
2. Development of a mold, into two halves, with two pins which make two holes for regulator cylinders and, at same time, these pins hold the metal structure in the over-injection.
3. Placing structures inside the mold.
4. Over-injection of soft material.
5. Demoulding of devices.

1. The fixer penile occluder device for urinary incontinence which consists of two stainless deformable articulate strips coated with soft material, with two holes to regulate the pressure, is characterized by having two elongated semicircular bulges in the top inside.
